(19) United States
(12) Patent Application Publication
(10) Pub. No.: US 2017/0227755 A1
(43) Pub. Date: Aug. 10, 2017

(54) OPTICAL SCANNING ENDOSCOPE APPARATUS

(71) Applicant: OLYMPUS CORPORATION, Tokyo (JP)

(72) Inventor: Keiichiro NAKAJIMA, Tokyo (JP)

(73) Assignee: OLYMPUS CORPORATION, Tokyo (JP)

(21) Appl. No.: 15/499,972

(22) Filed: Apr. 28, 2017

Related U.S. Application Data
(63) Continuation of application No. PCT/JP2014/005447, filed on Oct. 28, 2014.

(51) Int. Cl. G02B 23/24; G02B 26/10; A61B 1/06

(52) U.S. Cl. CPC G02B 23/2469; A61B 1/0638; A61B 1/0661; G02B 26/103; A61B 1/00172

(57) ABSTRACT

This optical scanning endoscope apparatus includes an actuator that scans light from a light source over an object with a predetermined scan cycle, a light amount detector that detects the light amount from the light source, and a controller that controls output of the light source based on the light amount detected by the light amount detector. During each scan cycle by the actuator, the controller controls the light source so as to output light according to a predetermined output change pattern, sequentially calculates an integral value of the light amount detected by the light amount detector over a predetermined time period, and controls the maximum of the change in output of the light source due to the output change pattern so that the integral value does not exceed a predetermined standard value.

### CROSS-REFERENCE TO RELATED APPLICATION

The present application is a Continuing Application based on International Application PCT/JP2014/005447 filed on Oct. 28, 2014, the content of which is incorporated herein by reference.

### TECHNICAL FIELD

This disclosure relates to an optical scanning endoscope apparatus for optically scanning an object.

### BACKGROUND

One known example of an optical scanning endoscope apparatus detects a luminance level based on reflected light from an object illuminated with light and controls the amount of illumination light in accordance with scanning position by setting the amount of illumination light so that, in the observation image, the light amount is reduced as the luminance level is brighter at the scanning position and is increased as the luminance level is darker at the scanning position (for example, see JP 2010-115391 A (PTL 1)).

### CITATION LIST

### Patent Literature

PTL 1: JP 2010-115391 A

### SUMMARY

An optical scanning endoscope apparatus according to this disclosure comprises:

a scanner configured to scan light from a light source over an object with a predetermined scan cycle;

a light amount detector configured to detect a light amount from the light source; and

a controller configured to control output of the light source based on the light amount detected by the light amount detector;

such that during each scan cycle by the scanner, the controller controls the light source so as to output light according to a predetermined output change pattern, sequentially calculates an integral value of the light amount detected by the light amount detector over a predetermined time period, and controls a maximum of a change in output of the light source due to the output change pattern so that the integral value does not exceed a predetermined standard value.

The controller preferably controls the light source so as to lower the maximum of the change in output of the light source due to the output change pattern when the integral value exceeds a first control threshold set to a value lower than the standard value.

When scanning a predetermined region of the object, the controller preferably controls the light source in accordance with the output change pattern to increase output of the light source more than when scanning a region other than the predetermined region. In this case, the optical scanning endoscope apparatus preferably further comprises an input interface configured to accept input to set the predetermined region of the object.

Furthermore, the scanner may scan light from the light source over a spiral scan path in a longitudinal direction on an inside of the object, the object being tubular; and when scanning a central region of the spiral scan path, the controller may control the light source in accordance with the output change pattern to increase output of the light source more than when scanning a peripheral region of the spiral scan path.

Alternatively, the scanner may scan light from the light source over a spiral scan path towards the object; and when scanning a peripheral region of the spiral scan path, the controller may control the light source in accordance with the output change pattern to increase output of the light source more than when scanning a central region of the spiral scan path.

The light source may be capable of emitting light of a plurality of wavelengths; and the controller may control the light source in accordance with the output change pattern to increase output of the light source for light of a particular wavelength among the plurality of wavelengths more than for light of other wavelengths.

Furthermore, the optical scanning endoscope apparatus further comprises a detector configured to detect light obtained from the object by scanning with light from the light source; the controller may control the light source in accordance with the output change pattern, the output change pattern being determined depending on a detection signal from the detector.

The standard value is determined based on safety standards for laser products.

The optical scanning endoscope apparatus may further comprise the light source, and the light amount detector may be structured integrally with the light source.

The controller preferably control the light source so as to raise the maximum of the change in output of the light source due to the output change pattern when the integral value of the light amount falls below a second control threshold lower than the first control threshold.

### BRIEF DESCRIPTION OF THE DRAWINGS

In the accompanying drawings:

FIG. 1 is a block diagram schematically illustrating the structure of an optical scanning endoscope apparatus according to Embodiment 1;

FIG. 2 is a schematic overview of the scope in FIG. 1;

FIG. 3 is a cross-sectional diagram of the tip of the scope in FIG. 2;

FIG. 4A is a side view, and FIG. 4B is a cross-sectional diagram along the A-A line in FIG. 4A, illustrating the vibration driving mechanism of the actuator and the oscillating portion of the light transmission fiber in FIG. 3;

FIG. 5 illustrates the vibration waveform in the X direction of the light transmission fiber;

FIG. 6 illustrates a spiral scan path;

FIG. 7 is a block diagram schematically illustrating the structure of the light amount detector in FIG. 1;

FIGS. 8A, 8B, 8C, and 8D illustrate operations by the light amount detector and the controller in FIG. 1;

FIGS. 9A to 9C illustrate an example of operations by the optical scanning endoscope apparatus according to Embodiment 1, where FIG. 9A illustrates the change over time in the scanning amplitude of the light transmission fiber, FIG. 9B illustrates the change in output of light from the light source, and FIG. 9C illustrates the change in the integral value, over a predetermined time period, of the light amount detected by the light amount detector;

FIGS. 10A, 10B, 10C, and 10D illustrate an example of operations by the light amount detector and the controller during a time period T_{X} that is a portion of the graph in FIGS. 9A to 9C;

FIGS. 11A to 11C illustrate modifications to the pattern of the change in output of the light source, where FIG. 11A is an output change pattern in which output at the central region of the spiral scan path is increased, FIG. 11B is an output change pattern in which output in a specific region is increased, and FIG. 11C is an output change pattern in which only the output of light with a specific wavelength is increased; and

FIGS. 12A to 12C illustrate a modification to the driver in FIG. 4, where FIG. 12A is a cross-sectional diagram of the tip of the scope, FIG. 12B is an enlarged perspective view of the driver in FIG. 12A, and FIG. 12C is a cross-sectional view along a plane perpendicular to the axis of the optical fiber, illustrating a portion including the coils for generation of a deflecting magnetic field and the permanent magnet in FIG. 12B.

### DETAILED DESCRIPTION

In general, considering the effect of laser light on the human eye and skin, a device that emits laser light is required, under JIS standards or the like, not to emit an amount of laser light exceeding a standard value within a certain time period (for example, 0.25 seconds).

In order for the light amount emitted to an object not to exceed a standard value, the maximum output of the light source could be set in advance so as not to exceed the standard value even when continuously emitting a constant light amount. With this approach, however, even when it is preferable to vary the output of light from the light source over time in conjunction with the scan cycle, the peak of the variable output becomes the set maximum output. Therefore, the light amount from the light source integrated over a certain time period falls far below the standard value, and the range of the light amount as required by the standard cannot be effectively used.

In embodiments of this disclosure, optical scanning endoscope apparatuses, while limiting the integral light amount from the light source irradiated within a certain time period to be below the standard value, allow observation that effectively uses the light amount from the light source allowed within the standard value.

Embodiments are described below with reference to the drawings.

### Embodiment 1

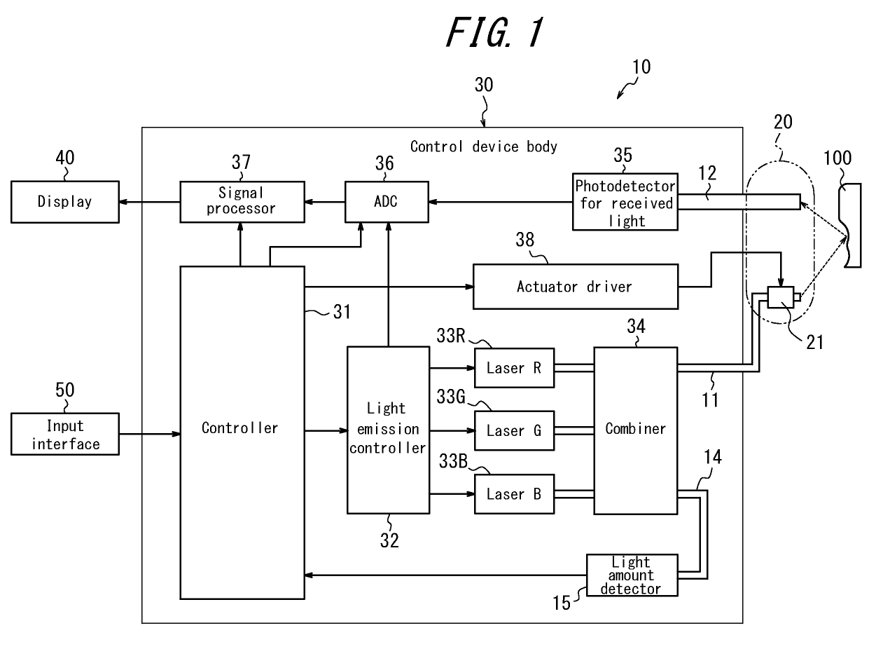

With reference to FIGS. 1 to 11C, Embodiment 1 is described. FIG. 1 is a block diagram schematically illustrating the structure of an optical scanning endoscope apparatus according to Embodiment 1. In FIG. 1, an optical scanning endoscope apparatus 10 includes a scope 20, a control device body 30, a display 40, and an input interface 50.

First, the structure of the control device body 30 is described. The control device body 30 includes a controller 31 that controls the optical scanning endoscope apparatus 10 overall, a light emission controller 32, lasers 33R, 33G, and 33B (the lasers 33R, 33G, and 33B also being collectively referred to below as a “light source 33”), a combiner 34, an actuator driver 38, a photodetector 35 for received light (detector), an analog/digital converter (ADC) 36, a signal processor 37, a monitor fiber 14, and a light amount detector 15. The controller 31 can set a variety of information from an external source via the input interface 50 (keyboard, mouse, touch panel, or the like).

In accordance with control by the light emission controller 32, the light source 33 constituted by the lasers 33R, 33G, and 33B selectively emits light of a plurality of different wavelengths (in this embodiment, light of three wavelengths: Red, Green, and Blue). As used herein, “selectively emits light of a plurality of different wavelengths” refers to light of one wavelength selected by the light emission controller 32 being emitted at a timing selected by the light emission controller 32. For example, Diode-Pumped Solid-State (DPSS) lasers or laser diodes may be used as the lasers 33R, 33G, and 33B.

In response to a control signal from the controller 31, the light emission controller 32 controls the light emission timing of the light source 33. In this embodiment, during one scan, the light emission controller 32 switches the wavelength of the R, G, or B light from the light source 33 in a predetermined light emission order (in this example, in the order R, G, B) at constant time intervals (light emission cycle T_{E}).

As used here, “one scan” refers to one scan, in order to capture one image, from the starting point to the ending point of a predetermined scan path, such as a spiral. The scan cycle during continuous scanning, for example the cycle from when the starting point of the scan path is scanned until the starting point of the scan path is scanned again during the next scan, is referred to as the “scan cycle T_{S}.” Furthermore, the “light emission cycle T_{E}” does not refer to the light emission cycle of each of the lasers 33R, 33G, and 33B constituting the light source 33, but rather to the light emission cycle of light that is sequentially emitted from the light source 33.

The laser light emitted from the lasers 33R, 33G, and 33B passes through optical paths joined coaxially by the combiner 34 and is incident as illumination light on a light transmission fiber 11, which is a single-mode fiber. The combiner 34 also partitions, to the light amount detector 15, a certain proportion of the output for the light transmission fiber 11. Since this proportion is nearly unaffected over time, a reduction in the accuracy of measurement, by the light amount detector 15, of the light amount is suppressed.

The combiner 34 may, for example, be configured using a fiber multiplexer, a dichroic prism, or the like.

The lasers 33R, 33G, and 33B and the combiner 34 may be stored in a housing that is separate from the control device body 30 and is joined to the control device body 30 by a signal wire.

Light incident on the light transmission fiber 11 from the combiner 34 is guided to the tip of the scope 20 and irradiated onto an object 100. At this time, by driving the actuator 21 of the scope 20 by vibration, the actuator driver 38 of the control device body 30 drives the tip of the light transmission fiber 11 by vibration. As a result, the illumination light emitted from the light transmission fiber 11 scans the observation surface of the object 100 in 2D over a predetermined scan path. Light such as reflected light or scattered light that is obtained from the object 100 due to irradiation with the illumination light is received at the tip of a light-receiving fiber 12, which is constituted by multi-mode fibers, and is guided through the scope 20 to the control device body 30.

In this example, the light transmission fiber 11 and the actuator 21 constitute a scanner that scans light from the light source 33 over the object 100.

The photodetector 35 for received light detects light from the object 100 through the light-receiving fiber 12, the light being obtained by irradiation of light at the wavelength (also referred to below as the color) of one of R, G, and B in each light emission cycle T_{E} of the light source 33 and outputs an analog signal (electrical signal).

The ADC 36 converts the analog signal from the photodetector 35 for received light to a digital signal (electrical signal) and outputs the result to the signal processor 37.

The signal processor 37 associates the digital signals, which correspond to the various wavelengths and were input from the ADC 36 in each light emission cycle T_{E}, with the respective light emission timings and scanning positions, and stores the results sequentially in memory (not illustrated). Information on the light emission timing and scanning position is acquired from the controller 31. The controller 31 calculates information on the scanning position along the scan path from information such as the amplitude and phase of vibration voltage applied by the actuator driver 38. After completion of scanning or during scanning, the signal processor 37 generates an image signal while performing image processing as necessary, such as enhancement, γ processing, and interpolation, based on each digital signal input from the ADC 36 and displays an image of the object 100 on the display 40.

The monitor fiber 14 is an optical fiber connecting the combiner 34 with the light amount detector 15 and guides, to the light amount detector 15, a certain proportion of the light output for the light transmission fiber 11 from the combiner 34.

The light amount detector 15 detects the light amount from the light source 33 and notifies the controller 31 of the detected light amount. As described below, the controller 31 sequentially calculates the integral value I of the light amount detected by the light amount detector 15 over the immediately prior predetermined integration period T_{A} and controls the light source 33 based on this calculated integral value I of the light amount.

Further details on the light amount detector 15 are provided below.

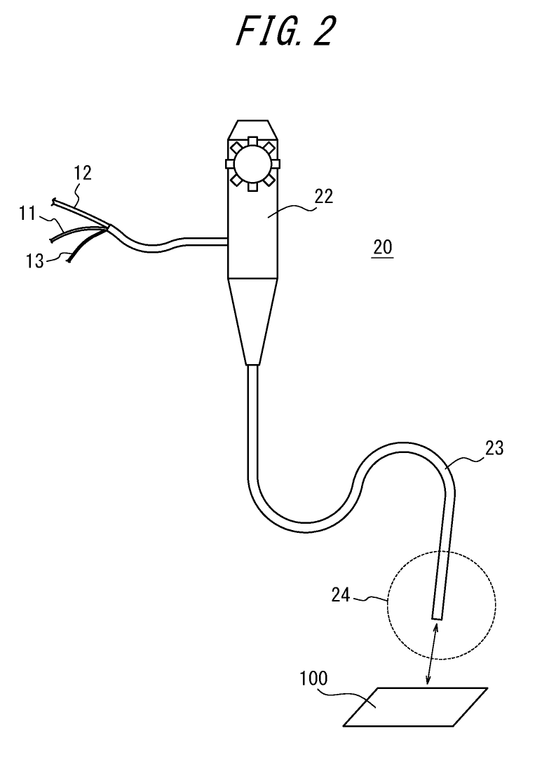

Next, the structure of the scope 20 is described. FIG. 2 is a schematic overview of the scope 20. The scope 20 includes an operation part 22 and an insertion part 23. The light transmission fiber 11, the light-receiving fiber 12, and a wiring cable 13 that extend from the control device body 30 are each connected to the operation part 22. The light transmission fiber 11, light-receiving fiber 12, and wiring cable 13 pass through the insertion part 23 and extend to a tip 24 (the portion within the dotted line in FIG. 2) of the insertion part 23.

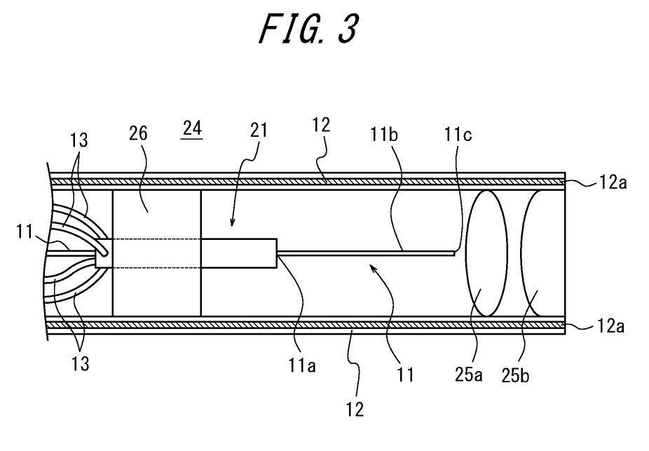

FIG. 3 is a cross-sectional diagram illustrating an enlargement of the tip 24 of the insertion part 23 of the scope 20 in FIG. 2. The tip 24 of the insertion part 23 of the scope 20 includes the actuator 21, projection lenses 25a and 25b (optical system), the light transmission fiber 11 that passes through the central portion, and the light-receiving fiber 12 that passes through the peripheral portion and is constituted by an optical fiber bundle.

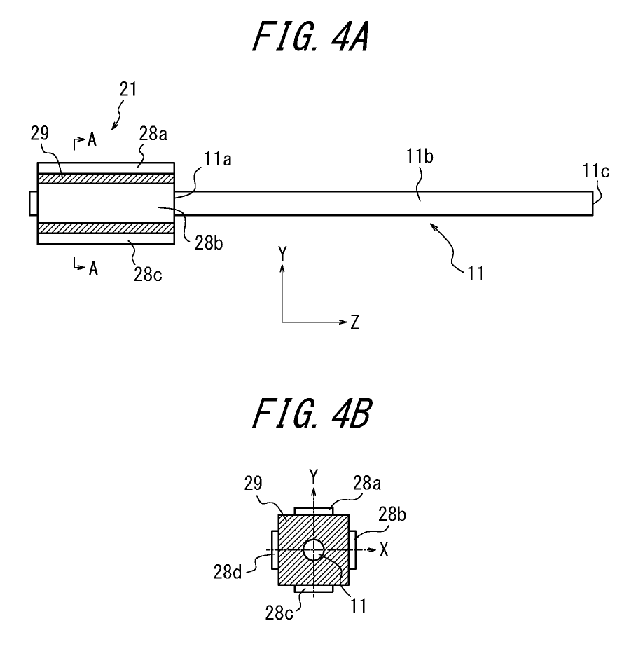

The actuator 21 drives a tip 11c of the light transmission fiber 11 by vibration. The actuator 21 includes a fiber holding member 29 fixed to the inside of the insertion part 23 of the scope 20 by an attachment ring 26 and piezoelectric elements 28a to 28d (see FIGS. 4A and 4B). The light transmission fiber 11 is supported by the fiber holding member 29, and the portion from a fixed end 11a supported by the fiber holding member 29 to the tip 11c is an oscillating part 11b that is supported to allow oscillation. The light-receiving fiber 12 is disposed to pass through the peripheral portion of the insertion part 23 and extends to the end of the tip 24. In some cases, a non-illustrated detection lens is also provided at the tip of each fiber in the light-receiving fiber 12.

Furthermore, the projection lenses 25a and 25b and the detection lenses are disposed at the extreme end of the tip 24 of the insertion part 23 in the scope 20. The projection lenses 25a and 25b are configured so that laser light emitted from the tip 11c of the light transmission fiber 11 is irradiated on the object 100 and roughly concentrated. The detection lenses are disposed so as to capture light that is reflected, scattered, or the like by the object 100 due to laser light concentrated on the object 100 or florescent light generated by irradiation of laser light concentrated on the object 100 (light obtained from the object 100), to concentrate the light on the light-receiving fiber 12 disposed behind the detection lenses, and to combine the light. The projection lenses are not limited to a double lens structure and may be structured as a single lens or as three or more lenses.

FIG. 4A illustrates the vibration driving mechanism of the actuator 21 of the optical scanning endoscope apparatus 10 and illustrates the oscillating part 11b of the light transmission fiber 11. FIG. 4B is a cross-sectional diagram along the A-A line in FIG. 4A. The vibration driving mechanism includes the piezoelectric elements 28a to 28d and the fiber holding member 29. The light transmission fiber 11 passes through the center of the fiber holding member 29, which is shaped as a quadratic prism, and is fixed and held by the fiber holding member 29. The four sides of the fiber holding member 29 respectively face the ±Y direction and the ±X direction. A pair of piezoelectric elements 28a and 28c for driving in the Y direction are fixed onto the sides of the fiber holding member 29 in the ±Y direction, and a pair of piezoelectric elements 28b and 28d for driving in the X direction are fixed onto the sides in the ±X direction.

The wiring cable 13 from the actuator driver 38 of the control device body 30 is connected to the piezoelectric elements 28a to 28d, which are driven by application of voltage by the actuator driver 38.

The pair of piezoelectric elements 28b and 28d in the X direction may, for example, be piezoelectric elements with the same direction of expansion and contraction relative to the application direction of voltage, and voltage of equivalent magnitude and opposite sign may always be applied. One of the piezoelectric elements 28b and 28d disposed opposite each other with the fiber holding member 29 therebetween expands and the other contracts, thereby causing the fiber holding member 29 to flex. Repeating this operation produces vibration in the X direction. The same is true for vibration in the Y direction as well.

The actuator driver 38 can perform vibration driving of the piezoelectric elements 28b and 28d for driving in the X direction and the piezoelectric elements 28a and 28c for driving in the Y direction by applying vibration voltage of the same frequency or vibration voltage of different frequencies thereto. Upon vibration driving of the piezoelectric elements 28a and 28c for driving in the Y direction and the piezoelectric elements 28b and 28d for driving in the X direction, the oscillating part 11b of the light transmission fiber 11 illustrated in FIGS. 3, 4A, and 4B vibrates, and the tip 11c is deflected, so that the laser light emitted from the tip 11c sequentially scans the surface of the object 100 over a predetermined scan path.

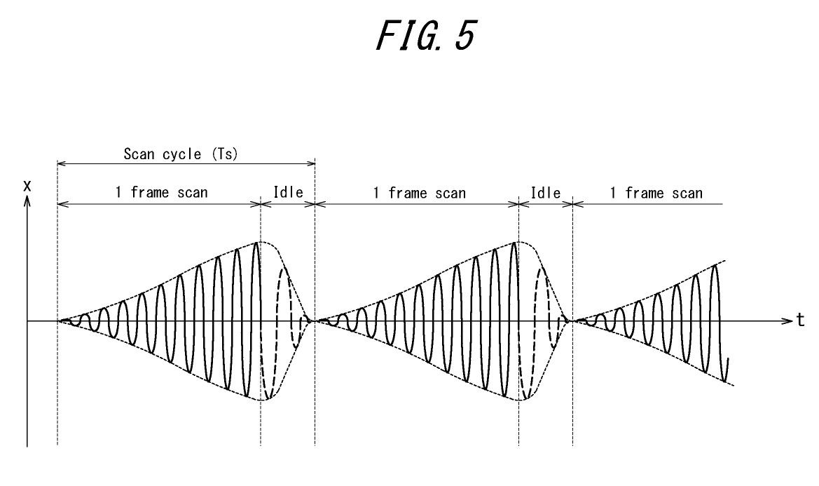

In this embodiment, with the aforementioned vibration driving mechanism, the object 100 is scanned over a spiral scan path. During each scan, a vibration voltage for vibration in a predetermined cycle starting from an amplitude of 0 while expanding to a predetermined maximum is applied to the piezoelectric elements 28b and 28d for driving in the X direction. As a result, the tip of the light transmission fiber 11 vibrates in a vibration waveform as illustrated by the solid line in FIG. 5 in the X direction. The amplitude of the vibration waveform of the fiber in FIG. 5 (which equals the value in the positive direction of the envelope shown by the dashed line in FIG. 5) is referred to as the scan amplitude A. At the same time that the vibration voltage is applied to the piezoelectric elements 28b and 28d for driving in the X direction, vibration voltage with the same cycle and amplitude as the vibration voltage for driving the piezoelectric elements 28b and 28d but shifted 90° in phase is applied to the piezoelectric elements 28a and 28c for driving in the Y direction. When the amplitude reaches its maximum, application of voltage to the piezoelectric elements 28a to 28d is suspended, or voltage that is controlled so as to reduce the amplitude is applied, and the amplitude of the tip 11c of the light transmission fiber 11 diminishes rapidly. In this way, the light transmission fiber 11 repeatedly scans over a spiral scan path. The cycle of the scan is designated as the scan cycle T_{S}.

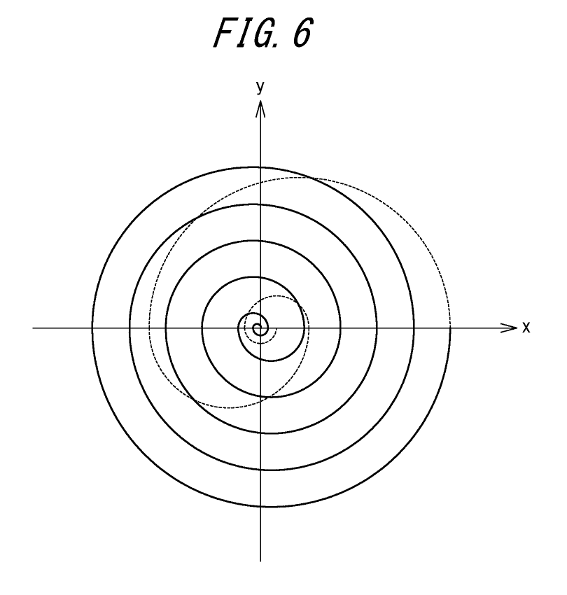

The controller 31 controls light emission of the lasers 33R, 33G, and 33B via the light emission controller 32 in synchronization with the driving of the tip 11c of the light transmission fiber 11 by the actuator driver 38. The lasers 33R, 33G, and 33B are controlled to emit light as the amplitude is increasing, and after the amplitude reaches its maximum, to suspend light emission while the amplitude diminishes. By driving the tip 11c of the light transmission fiber 11 in this way, the illumination light emitted from the tip 11c scans the object 100 in a spiral scan path, as indicated by the solid line in FIG. 6. The dashed line in FIG. 5 indicates the scan path as the amplitude diminishes. FIG. 6 is only a conceptual diagram of a scan, and an actual scan path on an object is more densely arranged.

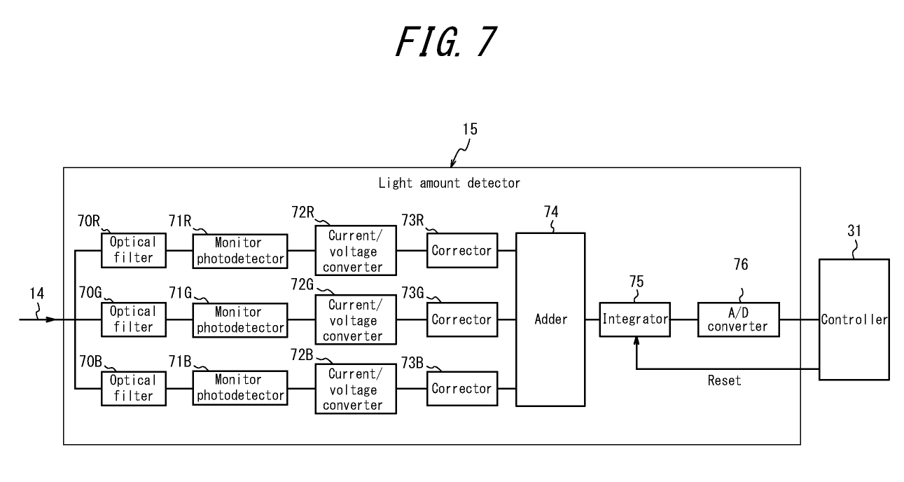

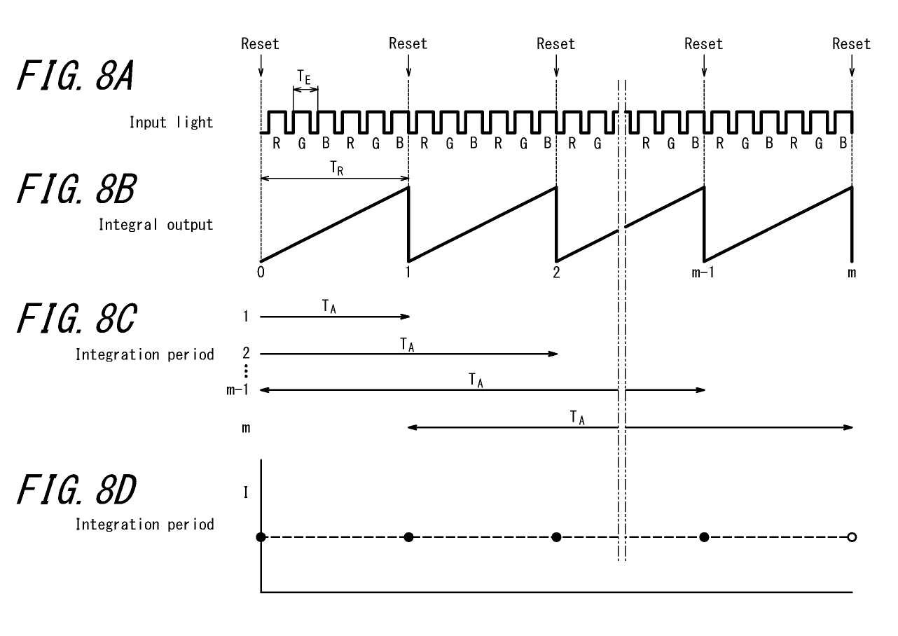

Next, with reference to FIG. 7 and FIGS. 8A to 8D, the light amount detector 15 is described in further detail. FIG. 7 schematically illustrates the structure of the light amount detector 15. FIGS. 8A to 8D illustrate operations by the light amount detector 15 and the controller 31. The light amount detector 15 includes optical filters 70R, 70G, and 70B, monitor photodetectors 71R, 71G, and 71B, current/voltage converters 72R, 72G, and 72B, correctors 73R, 73G, and 73B, an adder 74, an integrator 75, and an analog/digital (A/D) converter 76.

As illustrated in FIG. 8A, the optical filters 70R, 70G, and 70B divide up, by color, the R, G, and B light that is sequentially input from the monitor fiber 14 in each light emission cycle T_{E} of the light source 33 and output the divided R, G, and B light to the monitor photodetectors 71R, 71G, and 71B provided respectively for the colors R, G, and B. Since the output of light from the light source 33 in this embodiment is changed over time during the scan cycle T_{S}, the light that is input into the light amount detector 15 also changes over time. For the sake of explanation in FIG. 8A, however, the input light is illustrated as a row of pulses with a constant light amount.

The monitor photodetectors 71R, 71G, and 71B each detect light from the respective optical filters 70R, 70G, and 70B and output the detection result (current signal) to the current/voltage converters 72R, 72G, and 72B provided respectively for the colors R, G, and B.

The current/voltage converters 72R, 72G, and 72B convert the detection results (current signals) from the monitor photodetectors 71R, 71G, and 71B to respective voltage signals and output the voltage signals to the correctors 73R, 73G, and 73B provided respectively for the colors R, G, and B.

The correctors 73R, 73G, and 73B correct the respective detected signals (voltage signals) of R, G, and B light obtained from the monitor photodetectors 71R, 71G, and 71B via the current/voltage converters 72R, 72G, and 72B in accordance with each wavelength (color) of light and output the results to the adder 74.

In general, the light reception sensitivity of photodetectors such as the monitor photodetectors 71R, 71G, and 71B is dependent on wavelength.

Taking this into account, in the correctors 73R, 73G, and 73B, the detected signals (voltage signals) of R, G, and B light obtained from the monitor photodetectors 71R, 71G, and 71B via the current/voltage converters 72R, 72G, and 72B are corrected color by color so that the same voltage signal is obtained for input of the same light amount to the monitor photodetectors 71R, 71G, and 71B.

For example, when the monitor photodetectors 71R and 71B corresponding to R and B respectively output a 200 μA current signal based on 1 mW of R and B input light, and the monitor photodetector 71G corresponding to G outputs a 100 μA current signal based on 1 mW of G input light, then the light reception sensitivities of the monitor photodetectors 71R, 71G, and 71B corresponding to R, G, and B can be considered to be in a ratio of 2:1:2. In this case, the correctors 73R, 73G, and 73B corresponding to R, G, and B multiply the voltage signals input from the monitor photodetectors 71R, 71G, and 71B via the current/voltage converters 72R, 72G, and 72B respectively by factors of 1, 2, and 1 (i.e. only the corrector 73G corresponding to G doubles the input voltage signal), thus yielding the same voltage signals for the same input light amount.

By providing the correctors 73R, 73G, and 73B, the light amount from the light source 33 can be detected more accurately.

The detected signals of light of each color (voltage signals) corrected by the correctors 73R, 73G, and 73B respectively corresponding to R, G, and B are summed by the adder 74, and the result of summation is output to the integrator 75.

The integrator 75 is notified of a reset timing by the controller 31 at predetermined reset intervals T_{R} (for example, 0.001 seconds). As illustrated in FIG. 8B, upon reaching a reset timing, the integrator 75 starts to integrate the light detection signal input from the correctors 73R, 73B, and 73G via the adder 74, and upon reaching the next reset timing, outputs the result of integration over the immediately prior reset interval T_{R} to the A/D converter 76 as the light amount from the light source 33.

The A/D converter 76 converts the integration result from the integrator 75 to digital data by A/D conversion and notifies the controller 31 of the digital data as the light amount from the light source 33.

In each reset interval T_{R}, the controller 31 calculates the integral value I of the light amount, from the light source 33, detected over the immediately prior predetermined integration period T_{A} (for example, 0.25 seconds) by the light amount detector 15 (also referred to below simply as the “integral value I of the light amount”). In other words, as illustrated in FIG. 8C, in each reset interval T_{R}, the reference point of the start of integration shifts by the reset interval T_{R} (moving integration). The predetermined integration period T_{A} is set to be longer than the scan cycle T_{S}, and the reset interval T_{R} is set to be shorter than the scan cycle T_{S} (T_{A}>T_{S}>T_{R}). FIG. 8D illustrates the integral value I of the light amount calculated by the controller 31.

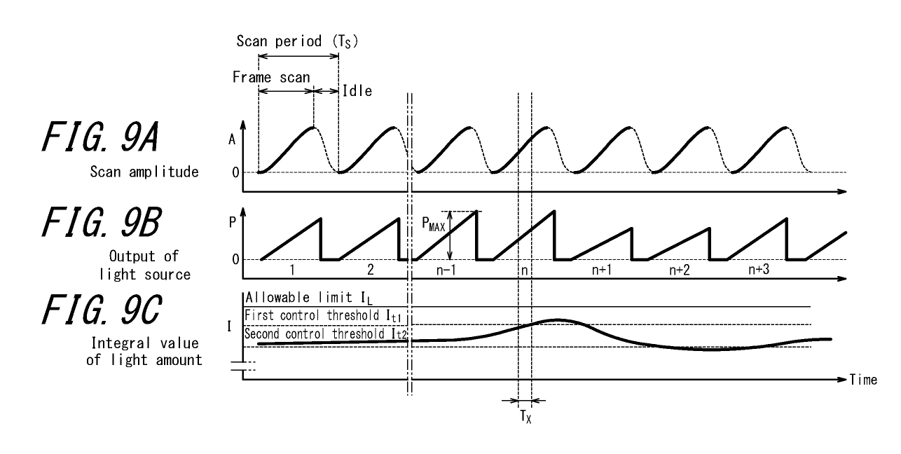

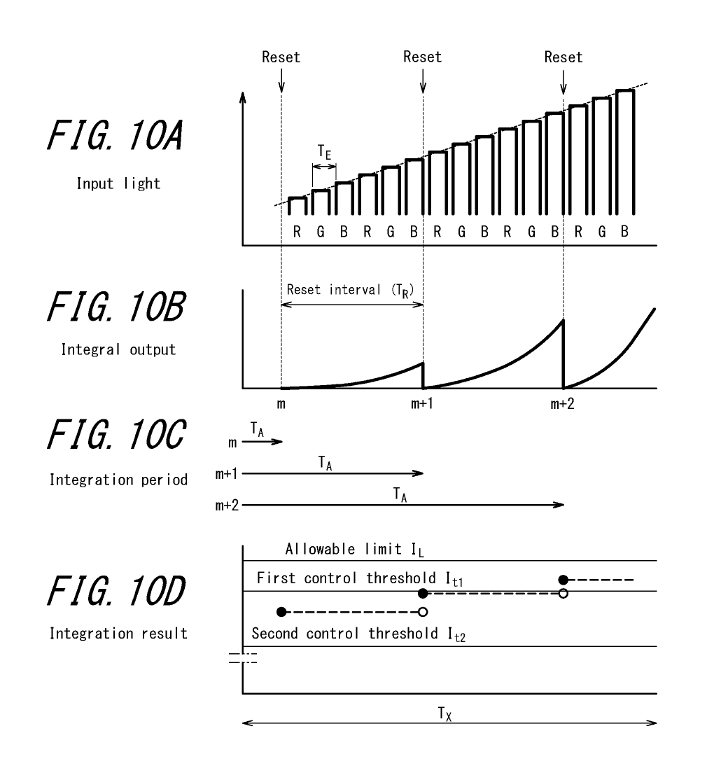

Next, FIGS. 9A to 9C illustrate an example of operations by the optical scanning endoscope apparatus according to this embodiment, where FIG. 9A illustrates the change over time in the scanning amplitude A of the light transmission fiber, FIG. 9B illustrates the change in output of light from the light source 33, and FIG. 9C illustrates the change in the integral value I, over a predetermined time period, of the light amount detected by the light amount detector 15. FIGS. 10A to 10D illustrate an example of operations by the controller 31 during a time period T_{X} that is a portion of the graph in FIGS. 9A to 9C.

As illustrated in FIG. 9A, the scan amplitude A of the light transmission fiber 11 gradually increases from 0 to the maximum during the scan cycle T_{S}. During this time, the object 100 is subjected to one frame scan from the central region to the outermost edge of the spiral scan. Subsequently, the scan amplitude A diminishes rapidly to zero. FIG. 9B illustrates the change over time in the output P of light from the light source 33 that is repeated in each scan cycle T_{S} (for example, 0.033 seconds) by the spiral scan. In FIG. 9B, reference numbers (1 to n+3) are indicated below the waves in the graph in correspondence with the scan cycle T_{S}. Here, the output P of light from the light source 33 changes over time by repeating a pattern in each scan cycle T_{S} such that the output P gradually increases from zero to the maximum P_{MAX} along with the increase in the scan amplitude A during one frame scan and then is set to 0 during the subsequent idle period. Such a pattern of change over time in the output of the light source 33, repeated in each scan cycle T_{S}, is referred to as the output change pattern. This “output change pattern” only stipulates the shape of the waveform for the change in output (the form of the increase or decrease in output) and does not include the amplitude of the change in output. On the other hand, the “output change” or “change in output” in this disclosure refers to the change over time in the magnitude of the output. The amplitude of the change in output of the light source 33, or the maximum output P_{MAX} when the minimum of the output is 0, is controlled by the controller 31. In other words, the controller 31 controls the maximum P_{MAX}, which is the amplitude of the waveform in the output change pattern, while causing output of light from the light source 33 to take the same output change pattern.

The output change pattern in FIG. 9B increases the output of the light source 33 while scanning from the central region to the periphery of the spiral scan path. When scanning over a spiral scan path, illumination light is irradiated at a greater inclination at the peripheral region than at the central region of the scan. Therefore, the intensity of reflected light or scattered light that is obtained from the object 100 tends to decrease at the peripheral region. Accordingly, in order to detect a uniform light amount across the entire scan range on the object 100, the output change pattern in FIG. 9B with an increased light amount from the light source 33 at the peripheral region is preferable.

On the other hand, when repeatedly scanning, the upper limit P_{MAX} on the change in output of the light source 33 is preferably set to as high a value as possible without the integral value I of the light amount exceeding the allowable limit I_{L}. As illustrated by the example in FIG. 9C, however, the integral value I of the light amount from the light source 33 over a predetermined time period may vary over time due to factors such as a change in room temperature.

Here, the controller 31 includes a first control threshold Iₜ₁ of the integral value I of the light amount detected by the light amount detector 15 over the predetermined integration period T_{A}. This first control threshold Iₜ₁ is set to a lower value than a predetermined allowable limit I_{L} (standard value) that the integral value I of the light amount is not supposed to exceed. The allowable limit I_{L} is the upper limit of the integral value I of the light amount per predetermined time period as allowed by standards such as JIS standards. In this embodiment, the controller 31 compares the integral value I of the light amount with the first control threshold Iₜ₁ at each reset interval T_{R} and controls the output of the light source 33 in each scan cycle T_{S} based on the result of comparison.

FIGS. 10A to 10D illustrate operations of the light amount detector 15 and the controller 31 during the time period T_{X} illustrated in FIGS. 9A to 9C when the light source 33 outputs light in the output change pattern illustrated in FIG. 9B. The time period T_{X} is selected as an example for illustration. FIG. 10A illustrates input light, similar to the input light illustrated in FIG. 8A, that is detected by the light amount detector 15. In this case, the intensity of input light increases as time elapses during one frame scan. FIG. 10B is the integrated output of light from the light source 33 output each reset interval T_{R}, as in FIG. 8B. The integrated output of light in each reset interval also increases along with the increase in input light. FIG. 10C illustrates a predetermined integration period T_{A} like FIG. 8C. Furthermore, like FIG. 8D, FIG. 10D illustrates the integral value I of the light amount calculated by the controller 31. Here, the integral value I of the light amount is obtained discreetly over time in each reset interval T_{R}. Whereas the integral value I of the light amount is a value integrated over the integration period T_{A} (for example, 0.25 seconds), the scan cycle T_{S} is sufficiently short (for example, 0.033 seconds). Therefore, when the light source 33 repeats light emission in each scan cycle T_{S} with the same output change pattern and within a range up to the constant maximum output P_{MAX}, the integral value I of the light amount averages out and does not vary greatly.

By contrast, as illustrated in FIG. 9B, the maximum output P_{MAX} in the output change pattern increases as time elapses, which may cause the integral value I of the light amount also to increase beyond the first control threshold Iₜ₁, as illustrated in FIG. 9C and FIG. 10D. Since the scale of time in FIGS. 9A to 9C is greater than in FIG. 10A to FIG. 10D, the integral value I of the light amount obtained intermittently at each reset interval T_{R} in FIG. 10D is illustrated by a continuous curve in FIG. 9C. As illustrated in FIG. 9C, for example when the controller 31 determines that the integral value I of the light amount has exceeded the first control threshold Iₜ₁ during the n^{th} scan cycle T_{S}, the controller 31 lowers the maximum output P_{MAX} of the light source 33 to suppress the output of light from the light source 33 in the scan with the output change pattern during the (n+1)^{th} and subsequent scan cycles T_{S}. The controller 31 suppresses the maximum P_{MAX} of the change in output of the light source 33 in such a way that the integral value I of the light amount does not exceed the allowable limit I_{L}.

In other words, during each scan cycle by the scanner, the controller 31 controls the light source 33 so as to output light according to a predetermined output change pattern and also sequentially calculates the integral value I of the light amount detected by the light amount detector 15 over a predetermined time period, controlling the maximum P_{MAX} of the change in output of the light source due to the output change pattern so that the integral value I of the light amount does not exceed the predetermined allowable limit I_{L}. Therefore, when the integral value I of the light amount exceeds the first control threshold Iₜ₁ set to a lower value than the allowable limit I_{L}, the controller 31 controls the light source 33 so as to lower the upper limit P_{MAX} on the output of the light source 33 in the output change pattern.

Also, after the controller 31 lowers the maximum P_{MAX} of the change in output of the light source 33 once, if the integral value I of the light amount falls below the second control threshold Iₜ₂, the controller 33 then raises the maximum P_{MAX} of the change in output of the light source 33 due to the output change pattern in the subsequent scan cycle T_{S} to cause the integral value I of the light amount to increase. For example, in FIG. 9C, since the integral value I of the light amount fell below the second control threshold Iₜ₂ during the (n+2)^{th} scan cycle T_{S}, the maximum P_{MAX} of the change in output of the light source 33 is increased in the (n+3)^{th} scan cycle T_{S}. In this way, the variation in the integral value I of the light amount can be kept within a certain range. For example, by setting the first control threshold Iₜ₁ to be 95% of the allowable limit I_{L} and the second control threshold Iₜ₂ to be 90% of the allowable limit I_{L}, the object 100 can continuously be irradiated with 90% or more of the allowable limit I_{L} and observed. The ratios of the first control threshold Iₜ₁ and of the second control threshold Iₜ₂ to the allowable limit I_{L} are set taking into consideration factors such as the output change pattern and the ratio of length between the integration period T_{A} and the scan cycle T_{S}.

According to this embodiment, the light amount detector 15 is provided, and the controller 31 monitors the light amount of the light source 33 and sequentially calculates the integral value I of the light amount over a predetermined time period, controlling the maximum P_{MAX} of the change in output of the light source 33 due to the output change pattern so that the integral value I of the light amount does not exceed the allowable limit I_{L} prescribed by standards for laser safety or the like. Therefore, the integral value I of the light amount from the light source 33 irradiated within a predetermined time period can be limited to be below the allowable limit I_{L}. Furthermore, since the maximum P_{MAX} of the change in output of the light source 33 is set based on the integral value I of the light amount, an optical scanning endoscope apparatus 10 that allows observation by effectively using the light amount of the light source 33 permitted within the allowable limit I_{L} can be provided. Also, the first control threshold Iₜ₁ and the second control threshold Iₜ₂ are provided, and with control by the controller 31, the maximum of the change in output of the light source 33 is lowered when the integral value I of the light amount exceeds the first control threshold Iₜ₁, and the maximum of the change in output of the light source 33 is raised when the integral value I of the light amount falls below the second control threshold Iₜ₂. Hence, the integral value I of the light amount can easily be kept within a desired range.

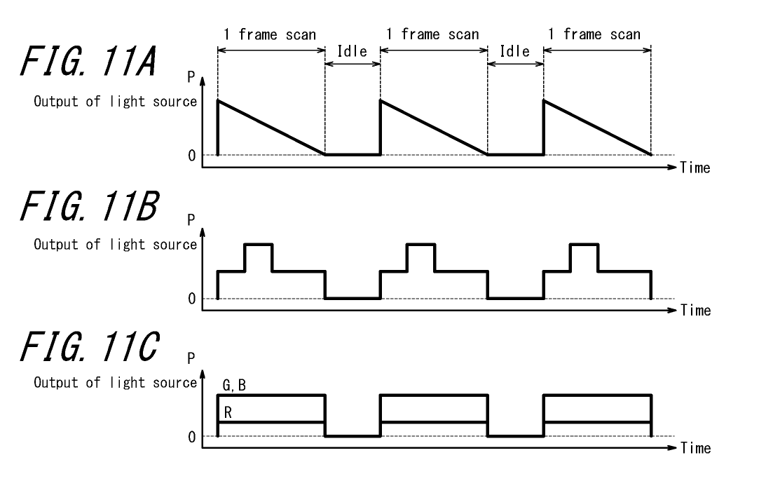

In this embodiment, an output change pattern that increases the light amount in the peripheral region of a spiral scan path is adopted, but another different output change pattern may be adopted instead. FIGS. 11A to 11C illustrate modifications to the output change pattern of the light source, where FIG. 11A is an output change pattern in which output of the light source 33 is made higher when scanning the central region of the spiral scan path than when scanning the peripheral region, FIG. 11B is an output change pattern in which output in a specific region is increased, and FIG. 11C is an output change pattern in which only the output of light with a specific wavelength is increased more than light of another wavelength. The following describes these output change patterns.

First, FIG. 11A is a suitable output change pattern when using a spiral scan path to observe a tubular object 100 in the longitudinal direction on the inside of the object 100. In this case, the object is closer further towards the peripheral region of the scan path, whereas at the central region of the scan path, either the object 100 is far away, or the illumination light does not reach the object 100. Accordingly, by outputting light from the light source 33 in the output change pattern illustrated in FIG. 11A, an image with more uniform brightness across the entire scan range can be obtained.

FIG. 11B is an output change pattern in which, when scanning a predetermined region on the object 100, the output of the light source 33 is increased more than when scanning a region other than the predetermined region. FIG. 11B is, for example, a scan path in a slow-scan direction during a raster scan, and the output of the light source 33 is set higher when scanning a predetermined region in the slow scan direction. Also, when combining with a scan in the fast scan direction, the output of the light source 33 can be increased when scanning a predetermined region of the object 100. For example, the user of the optical scanning endoscope apparatus 10 can set this predetermined region by setting a location with the input interface 50 (input interface) while confirming an image displayed on the display 40. With this approach, the user can specify a region of particular interest in the object 100 under observation and can acquire a clearer image. Devices in a variety of forms may be used as the input interface 50, such as a mouse, keyboard, touch panel display, or the like. When scanning the object 100 with a spiral scan path as well, the output of the light source 33 can be increased when scanning a predetermined region.

Furthermore, FIG. 11C is an output change pattern in which the output of the G and B colored light source 33 is increased, whereas R colored light is decreased. In this way, in accordance with optical characteristics of the object 100, the output of light of a particular color from the light source 33 can be increased over or decreased below the output of light of other colors. For example, when observing a blood vessel, the output change pattern of FIG. 11C that reduces the amount of red light is preferable. By reducing the amount of R light, the amount of G and B light can be increased within the range of the allowable limit I_{L} of the integral value of the light amount, thus yielding a brighter image.

This disclosure is not limited to the above embodiment, and a variety of modifications may be made. For example, the light amount detector 15 may be formed integrally with the light source 33 as a photodiode (PD). In this case, the light amount detector 15 is disposed on the upstream side of the combiner 34.

This disclosure is not limited to the case of scanning with a spiral scan path or scanning with a raster-shaped scan path and may also be applied to an optical scanning endoscope apparatus that scans using a so-called Lissajous pattern scan path. A variety of combinations of output change patterns and scan paths are possible.

Furthermore, in the above embodiment, the controller 31 controls the output of the light source 33 in accordance with a predetermined output change pattern, but the controller 31 may instead acquire a signal, detected by the photodetector 35 for received light, via the ADC 36 or the signal processor 37 and determine the output change pattern depending on this signal. For example, an output change pattern may be generated to increase the output of the light source 33 when scanning a region with a small detected light amount (reflected light, scattered light, or the like) obtained by the photodetector 35 for received light. By doing so, a region that would be dark in the display of the object 100 can be displayed more brightly.

Furthermore, in the example illustrated in FIG. 7, by providing the light amount detector 15 with the optical filters 70R, 70G, and 70B that divide light into R, G, and B light, correction can be made taking into consideration the light reception sensitivity of each color in the correctors 73R, 73G, and 73B even when light of a plurality of colors is input simultaneously, or when the light source 33 is a white light source. Hence, the light amount from the light source 33 can be calculated accurately.

In the case of the R, G, and B light being sequentially input into the light amount detector 15, the light amount detector 15 may, instead of including optical filters and an adder, be configured to include one each of a monitor photodetector, a current/voltage converter, a corrector, an integrator, and an A/D converter, and at the timing at which the R, G, and B light is sequentially input, the processing by the corrector may be switched in accordance with the color of light.

A level corrector (not illustrated) may also be provided between the correctors 73R, 73G, and 73B and the adder 74, and level correction may be performed on the signal in accordance with the irradiation distance to the object, irradiation position, and the like. Alternatively, without providing the correctors 73R, 73G, and 73B and the adder 74, a total of three each of an integrator and an A/D converter may be provided in association with light of R, G, and B wavelengths in the light amount detector 15 illustrated in FIG. 7, and the output from the current/voltage converters 72R, 72G, and 72B may be input into the controller 31 via the corresponding integrators and A/D converters. In this case, instead of using the correctors 73R, 73G, and 73B, the controller 31 may correct signals in accordance with the wavelength of light.

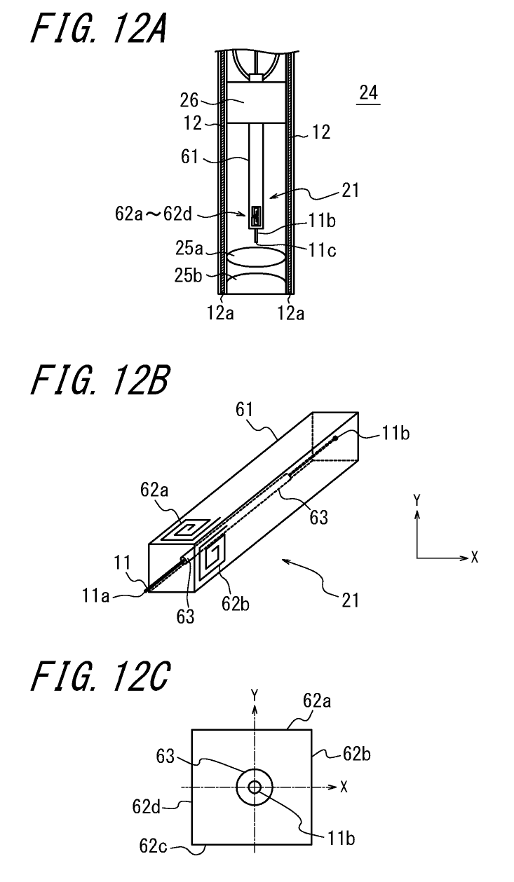

The actuator 21 of the light transmission fiber 11 is not limited to use of piezoelectric elements. For example, a permanent magnet fixed to the light transmission fiber 11 and coils for generation of a deflecting magnetic field (magnet coils) that drive the permanent magnet may be used instead. The following describes a modification to the actuator 21 with reference to FIGS. 12A to 12C. FIG. 12A is a cross-sectional diagram of the tip 24 of the scope 20, FIG. 12B is an enlarged perspective view of the actuator 21 in FIG. 12A, and FIG. 12C is a cross-sectional view along a plane perpendicular to the axis of the light transmission fiber 11, illustrating a portion including the coils 62a to 62d for generation of a deflecting magnetic field and the permanent magnet 63 in FIG. 12B.

At a portion of the oscillating part 11b of the light transmission fiber 11, the permanent magnet 63, which is magnetized in the axial direction of the light transmission fiber 11 and includes a through-hole, is joined to the light transmission fiber 11 by the light transmission fiber 11 being passed through the through-hole. A square tube 61, one end of which is fixed to the attachment ring 26, is provided so as to surround the oscillating part 11b, and flat coils 62a to 62d for generation of a deflecting magnetic field are provided on the sides of the square tube 61 at a portion thereof opposing one pole of the permanent magnet 63.

The pair of coils 62a and 62c for generation of a deflecting magnetic field in the Y direction and the pair of coils 62b and 62d for generation of a deflecting magnetic field in the X direction are each disposed on opposing sides of the square tube 61, and a line connecting the center of the coil 62a for generation of a deflecting magnetic field with the center of the coil 62c for generation of a deflecting magnetic field is orthogonal to a line connecting the center of the coil 62b for generation of a deflecting magnetic field with the center of the coil 62d for generation of a deflecting magnetic field near the central axis of the square tube 61 when the light transmission fiber 11 is disposed therein at rest. These coils are connected to the actuator driver 38 of the control device body 30 via the wiring cable 13 and are driven by drive current from the actuator driver 38.

Furthermore, the scanner is not limited to oscillating the tip of an optical fiber. For example, an optical scanning element such as a MEMS mirror may be disposed along the optical path from the light source 33 to the object.

### REFERENCE SIGNS LIST

10 Optical scanning endoscope apparatus
11 Light transmission fiber (scanner)
11a Fixed end
11b Oscillating part
11c Tip
12 Light-receiving fiber
13 Wiring cable
14 Monitor fiber
15 Light amount detector
20 Scope
21 Actuator (scanner)
22 Operation part
23 Insertion part
24 Tip
25a, 25b Projection lens
26 Attachment ring
28a to 28d Piezoelectric element
29 Fiber holding member
30 Control device body
31 Controller
32 Light emission controller
33 Light source
33R, 33G, 33B Laser
34 Combiner
35 Photodetector for received light
36 ADC
37 Signal processor
38 Actuator driver
40 Display
50 Input interface
61 Square tube
62a to 62d Coil for generation of a deflecting magnetic field
63 Permanent magnet
70R, 70G, 70B Optical filter
71R, 71G, 71B Monitor photodetector
72R, 72G, 72B Current/voltage converter
73R, 73G, 73B Corrector
74 Adder
75 Integrator
76 A/D converter
100 Object
T_{S} Scan cycle
T_{E} Light emission cycle
T_{R} Reset interval
T_{A} Integration period
I_{L} Allowable limit
Iₜ₁ First control threshold
Iₜ₂ Second control threshold
A Scan amplitude
P Output of light source
I Integral value of light amount

1. An optical scanning endoscope apparatus comprising:
a scanner configured to scan light from a light source over an object with a predetermined scan cycle;
a light amount detector configured to detect a light amount from the light source; and
a controller configured to control output of the light source based on the light amount detected by the light amount detector;
wherein during each scan cycle by the scanner, the controller controls the light source so as to output light according to a predetermined output change pattern, sequentially calculates an integral value of the light amount detected by the light amount detector over a predetermined time period, and controls a maximum of a change in output of the light source due to the output change pattern so that the integral value does not exceed a predetermined standard value.

2. The optical scanning endoscope apparatus of claim 1, wherein the controller controls the light source so as to lower the maximum of the change in output of the light source due to the output change pattern when the integral value exceeds a first control threshold set to a value lower than the standard value.

3. The optical scanning endoscope apparatus of claim 1, wherein when scanning a predetermined region of the object, the controller controls the light source in accordance with the output change pattern to increase output of the light source more than when scanning a region other than the predetermined region.

4. The optical scanning endoscope apparatus of claim 3, further comprising an input interface configured to accept input to set the predetermined region of the object.

5. The optical scanning endoscope apparatus of claim 1,
wherein the scanner scans light from the light source over a spiral scan path in a longitudinal direction on an inside of the object, the object being tubular; and
wherein when scanning a central region of the spiral scan path, the controller controls the light source in accordance with the output change pattern to increase output of the light source more than when scanning a peripheral region of the spiral scan path.

6. The optical scanning endoscope apparatus of claim 1,
wherein the scanner scans light from the light source over a spiral scan path towards the object; and
wherein when scanning a peripheral region of the spiral scan path, the controller controls the light source in accordance with the output change pattern to increase output of the light source more than when scanning a central region of the spiral scan path.

7. The optical scanning endoscope apparatus of claim 1,
wherein the light source is capable of emitting light of a plurality of wavelengths; and
the controller controls the light source in accordance with the output change pattern to increase output of the light source for light of a particular wavelength among the plurality of wavelengths more than for light of other wavelengths.

8. The optical scanning endoscope apparatus of claim 1, further comprising:
a detector configured to detect light obtained from the object by scanning with light from the light source;
wherein the controller controls the light source in accordance with the output change pattern, the output change pattern being determined depending on a signal from the detector.

9. The optical scanning endoscope apparatus of claim 1, wherein the standard value is determined based on safety standards for laser products.

10. The optical scanning endoscope apparatus of claim 1, further comprising the light source, wherein the light amount detector is structured integrally with the light source.

11. The optical scanning endoscope apparatus of claim 2, wherein the controller controls the light source so as to raise the maximum of the change in output of the light source due to the output change pattern when the integral value of the light amount falls below a second control threshold lower than the first control threshold.
